(19) United States
(12) Patent Application Publication
(10) Pub. No.: US 2005/0084533 A1
(43) Pub. Date: Apr. 21, 2005

(54) Polymer composite with internally distributed deposition matter

(76) Inventors: Steven Melvyn Howdle, Nottingham (GB); Kevin Morris Shakesheff, Nottingham (GB); Martin James Whitaker, Nottingham (GB); Michael Stephen Watson, Nottingham (GB)

Correspondence Address: JACOBSON HOLMAN PLLC, 400 SEVENTH STREET N.W., SUITE 600, WASHINGTON, DC, 20004, US

(21) Appl. No.: 10/506,618

(22) PCT Filed: Mar. 10, 2003
(86) PCT No.: PCT/GB03/01015

(30) Foreign Application Priority Data
Mar. 13, 2002 (GB) 0205868.3

(51) Int. Cl. A61K 9/14; C08L 1/00

(52) U.S. Cl. 424/486; 523/122

(57) ABSTRACT

A process for the preparation of a polymer composite comprising internally distributed deposition matter wherein the process comprises providing a deposit of deposition matter at the surface of a solid state polymer substrate, contacting the surface deposited polymer with a plasticising fluid or a mixture of plasticising fluids under plasticising conditions to plasticise and/or swell the polymer and internally distribute deposition matter, and releasing the plasticising fluid or fluids to obtain polymer composite.; A polymer composite comprising a porous or non porous polymer throughout which particulate deposition matter as hereinbefore defined is distributed with desired uniformity, preferably with high uniformity in excess of 80% for example in excess of 98%.; A scaffold comprising a polymer composite having internally distributed deposition matter; and use of the composite as a support or scaffold for drug delivery, for use in bioremediation, as a biocatalyst or biobarrier for human or animal or plant matter, for use as a structural component, for example comprising the polymer and optional additional synthetic or natural metal, plastic, carbon or glass fibre mesh, scrim, rod or like reinforcing for medical or surgical insertion, for insertion as a solid monolith into bone or tissue, as fillers or cements for wet insertion into bone or teeth or as solid aggregates or monoliths for orthopaedic implants such as pins, or dental implants such as crowns etc.

The present invention relates to a process for the preparation of a polymer composite comprising contacting polymer with plasticising fluid and deposition matter and isolating polymer comprising internally distributed deposition matter, the polymer composite obtained thereby, and apparatus for the preparation thereof, a polymer scaffold, drug delivery device or the like comprising the composite in suitably sized and shaped form, the use as a pharmaceutical or veterinary product, a human or animal health or growth promoting, structural, fragrance or cosmetic product, an agrochemical or crop protection product, in biomedical, catalytic and like applications, more particularly as a biodegradable slow release product, or as biodegradable surgical implant, synthetic bone composite, organ module, and the like or for bioremediation, as a biocatalyst or biobarrier and the like.

The use of supercritical fluids in the production of polymers as a plasticising, foaming or purification agent is known. Supercritical fluids (SCFs) act as plasticisers for many polymers, increasing the mobility of the polymer chains. This results in an increase in the free volume within the polymeric material.

Supercritical fluid has found application in incorporation of dyes and other inorganic materials which are insoluble in the supercritical fluid, for example inorganic carbonates and oxides, into polymers with a good dispersion to improve quality, in particular dispersion in products such as paints for spray coating and the like.

Moreover the fluid can be used to foam the polymer by transition to non-critical gaseous state whereby a porous material may be obtained and this has been disclosed in U.S. Pat. No. 5,340,614, WO91/09079 & U.S. Pat. No. 4,598,006.

U.S. Pat. No. 5,340,614 discloses simultaneously contacting polymer, impregnation additive and SCF. U.S. Pat. No. 4,598,006 discloses dissolving impregnation additive in SCF, adding polymer and releasing fluid with transition to subcritical conditions.

WO 91/09079 (De Ponti) discloses preloading polymer microspheres with an active ingredient such as a drug by dissolving polymer in solvent, adding a solution of active ingredient, and mixing in silicone oil to obtain loaded microspheres. These are washed and hardened. Microspheres are then SCF processed to produce a porous structure.

However the double emulsion process of WO 91/09079 has shown in some cases only 68% retained drug activity compared with control and this is attributed to solvent effects, homogenising the double emulsion, breaking up droplets and the like.

Moreover this process is quite complex, requiring two polymer processing stages, and does not necessarily ensure good internal distribution.

Polymers have also been used in biomedical applications to develop materials in which biocompatibility can be influenced to promote favourable tissue responses whilst also producing materials with acceptable mechanical and surface properties. Biofunctional composite materials e.g. calcium hydroxyapatite dispersed in various polymers are well established for orthopaedic, dental and other applications. These materials are prepared with very high loadings of inorganic solid, of up to 80%, in the form of a powder, and a composite is formed either by vigorous mixing of the powdered material into the solid or molten polymer, or by polymerisation of the monomers in the presence of suspended inorganic powders. In both cases, the material becomes entrapped within the polymer matrix.

These methods for preparation however are prone to insufficient and uncontrolled mixing of material leading to large aggregate formation whereby the composite is prone to fracture and may not be suitable for commercial processing.

WO 98/51347 (Howdle et al) discloses the preparation by dense phase fluid processing of biofunctional polymers comprising biofunctional material having the desired mechanical properties both for commercial processing and for implant into a human or animal host structure such as bone or cartilage, dental and tissue structures into which they are surgically implanted for orthopaedic bone and implant, prosthetic, dental filling or restorative applications, prolonged release applications and the like. Biofunctional material is in particular any pharmaceutical, veterinary, agrochemical, human and animal health and growth promoting, structural, cosmetic and toxin absorbing materials, such as a broad range of inorganic or organic molecules, peptides, proteins, enzymes, oligosaccharides, carbohydrates, nucleic acids and the like.

Particular application is in the production of bone composites formed from a biofunctional polymer with inorganic calcium hydroxyapatite uniformly distributed throughout. This process uses the addition of CO₂ to plasticise polymeric material and highly efficient stirring to ensure homogeneous incorporation of particulate material throughout the polymer.

This and other work from the same authors has shown high uniformity. However there is a need for further improved uniformity for both high and low loading levels, with milder processing conditions. Therapeutic concentrations of growth factors and other biotechnology drugs are of the order of ppb, whilst those of biocompatibilisers such as hydroxyapatite are of the order of 80 wt %. Greater uniformity manifests itself in more uniform prolonged release, and stronger monolithic structures.

We have now surprisingly found that controlled internal distribution of matter within a polymer composite can be achieved in a simple and reproducible process, which enables the accurate and efficient handling of biologically active molecules in small or large amount in solution while retaining the manifold advantages of SCF processing. The present invention provides deposition of matter on a polymer surface in a first stage and internal distribution and optional pore formation in a second polymer plasticisation stage. This is in contrast to WO 91/09079 which teaches dissolving polymer and emulsifying with impregnation matter in a first stage, and plasticising in a second stage.

Accordingly in the broadest aspect of the invention there is provided a process for the preparation of a polymer composite comprising internally distributed deposition matter wherein the process comprises providing a deposit of deposition matter at the surface of a solid state polymer substrate, contacting the surface deposited polymer with a plasticising fluid, or a mixture of plasticising fluids under plasticising conditions to plasticise and/or swell the polymer and internally distribute deposition matter, and releasing the plasticising fluid or fluids to obtain polymer composite.

Preferably the process comprises providing a deposit at the surface of a high surface area polymer substrate, more preferably a powder bed or a high porosity matrix. Preferably the process provides a deposition layer of deposition matter on the internal and external surfaces of the polymer substrate, more preferably any exposed surfaces, including any exposed surface pores. By this means a more dilute deposit is formed which is of greater uniformity than depositing the same quantity of material on a smaller surface area. Deposition may be over the entire surface area or only part or parts thereof.

Preferably a porous solid state polymer substrate is obtained by contacting polymer with plasticising fluid and subsequently releasing fluid in suitable manner to foam the polymer as is known in the art. In a preferred embodiment therefore the process comprises in a first stage contacting polymer with plasticising fluid or a mixture of plasticising fluids under plasticising conditions to plasticise the polymer, and releasing the fluid to obtain a solid state substrate polymer; in a second stage providing a surface deposit of deposition matter at the surface of the polymer, and in a third stage contacting the surface deposited polymer with a plasticising fluid or a mixture of plasticising fluids under plasticising conditions to plasticise and/or swell the polymer and internally distribute deposition matter, and releasing the plasticising fluid or fluids to obtain polymer composite. Preferably in the first stage the plasticising and releasing the fluid(s) is in manner to foam the polymer and obtain a porous solid state substrate polymer, for use in the second stage.

The product composite may be porous or non-porous, even if obtained from a porous substrate. It is a particular advantage that porosity may serve to facilitate surface deposition, but be of little interest in the product composite or vice versa or a combination thereof.

Deposition may be of discrete particles or of dissolved deposition matter and may be by solid or fluid phase deposition. Preferably deposition matter is provided in fluid phase, and deposition comprises immersion, spraying and the like with a solution, dispersion or suspension of deposition matter and drying by freezing, evaporation, heating, blotting etc.

Alternatively deposition matter is provided in solid phase and deposition comprises powder coating, dusting, rolling or adhering.

Deposition may be aided by softening or adhesion of surface polymer, in particularly in the case of deposition of insoluble or dry phase deposition matter.

Deposition may be with or without physical interaction with the polymer surface. In a particularly preferred embodiment, on contacting polymer substrate with a solution, dispersion or suspension of deposition matter, the deposition matter adsorbs from liquid phase onto the polymer surface and forms an adsorption layer of deposition matter at desired levels. This layer remains intact to solvent and impact effects and the like, for example if subsequently surface washed with liquids.

Immersion time may be of the order 1 second up to 48 hours, depending on the materials used. Drying time may be up to 48 hours depending on sensitivity to extreme heat or freezing or the like.

Preferably deposition matter is provided in particulate or powder form and may be of particle size in the range up to 1 mm, preferably 50-1000 micron. Deposition matter may be of uniform or mixed particle size, depending on practical constraints and the required distribution, and may be of same or different matter.

The polymer is suitably in the solid phase or is a highly viscous fluid and may present limited or good mixing characteristics. Solid phase polymer may be particulate, eg in the form of granules, pellets, microspheres, powder, or monolithic eg matrix form. Plasticising conditions comprise conditions of reduced viscosity to plasticise and/or swell the polymer. It is known that particulate polymer agglomerates on plasticisation to a larger structure. This may revert to a particulate composite or form a monolithic composite on release of plasticising fluid, as hereinbelow defined. Polymer volumes of 5 or 10 mg or g up to multi kg scale may be used.

Reference herein to a plasticising fluid is to a fluid which is able to plasticise polymer in its natural state or in supercritical, near critical, dense phase or subcritical state. Fluid may be liquid or gaseous, and is preferably selected for a suitable density which is capable of plasticising a given polymer, fluid density may be in the range 0.001 g/ml up to 10 g/ml for example 0.001 g/ml up to 2 g/ml.

Plasticising conditions comprises elevated or ambient temperature, and/or elevated or ambient pressure. Fluid may be selected for effective plasticisation of a given polymer under conditions which are amenable to the deposition matter or alternatively fluid is selected by preferred chemical type and suitable plasticising conditions are chosen for that fluid, preferably selecting a first amenable condition (T) and compensating with second condition (P) to obtain desired density.

Preferably the plasticising conditions comprise a desired temperature less than, equal to or greater than the fluids critical temperature (Tc) in the range −200° C. to +500° C., preferably −200° C. to 200° C., more preferably −100 to +100° C., for example −80 or −20° C. to +200 or +100° C. For most fluids this will be in the range approximately 10 to 15° C., 15 to 25° C., 25 to 30° C., 30 to 35° C., 35 to 45° C. or 45 to 55° C., most preferably approximately 28 to 33° C. (CO₂). Other sub ranges may be envisaged and are within the scope of the invention. Preferably the lowest temperature is employed which is compatible with sufficient lowering of the polymer Tg to achieve plasticisation. To operate at ambient temperature, the process of the invention may require compensation by increase in pressure.

Preferably the plasticising fluid comprises a desired pressure less than, equal to or greater than the plasticising fluids critical pressure (Pc) from in excess of 1 bar to 10000 bar, preferably 1 to 1000, more preferably 2 to 800 bar, more preferably 2 to 400 bar, more preferably 5 to 265 bar, most preferably 15 to 75 bar. For most fluids this will be in the range approximately 30 to 40 bar, 40 to 50 bar, 50 to 60 bar, 60 to 75 bar or 80 to 215 bar, and is most preferably approximately 34 to 75 bar for dense phase or supercritical CO₂. Other sub ranges may be envisaged and are within the scope of this invention.

Fluid may be provided at plasticising conditions prior to contacting with polymer and deposition matter or may be brought to plasticising conditions in contact with surface deposited polymer.

Preferably the process is carried out for a contact time of surface deposited polymer and plasticising fluid of 1 millisecond up to 5 hours. Short contact time may be preferred for example 2 milliseconds up to 10 minutes, more preferably 20 milliseconds to 5 minutes, more preferably 1 second to 1 minute, more preferably 2 to 30 seconds, most preferably 2 to 15 seconds. Alternatively long contact time minimises detrimental effects of pressurising the vessel, and allows superior distribution, for example 15 minutes to 2 hours, preferably 15 minutes to 40 minutes or 30 minutes to 1 hour.

Pressurising plasticising fluid may be in situ, or ex situ prior to contacting with surface deposited polymer as hereinbefore defined. The pressurisation period whereby in the case of in situ or ex situ pressurisation the fluid is pressurised or is introduced to the surface deposited polymer, is suitably for a period of 1 second to 3 minutes, more preferably from 1 second to 1 minute, more preferably from 1 to 45 seconds.

The process may be carried out with or without stirring or blending. Blending and conditions may be selected to assist plasticisation or according to the desired uniformity and distribution of loading. In the case that uniform distribution is required the process preferably comprises blending for prolonged period and/or high intensity. In the case that non-uniform distribution is envisaged, the process may be carried out simply with stirring.

Blending may be by physical mixing, pumping, agitation for example with aeration or fluidising gas flow, lamellar flow or otherwise impregnation or diffusion of plasticising fluid throughout the surface deposited polymer. Stirring is typically with use of stirrers and impellers, preferably helical impellers such as helical ribbon impellers for enhanced blending and the like.

Blending may be for a period of 1 millisecond up to 5 hours and may be for the duration of contacting with plasticising fluid or otherwise. Preferably stirring or blending is for substantially the duration of contacting with plasticising fluid, with period of stirring or blending corresponding to period of plasticising fluid contacting as hereinbefore defined.

The process comprises subsequently releasing the plasticising fluid. In the case that plasticising conditions comprises elevated pressure release is under reduced pressure conditions, conducted over a desired depressurisation period, whereby the polymer composite is obtained comprising internally distributed deposition matter. Depressurisation may be achieved in situ, by depressurising a pressure vessel in which the process is carried out, whereby a monolithic block of polymer composite is obtained. Alternatively the contents of a pressure vessel in which the process is conducted may be discharged into a second pressure vessel at lower pressure whereby a homogeneous powder of polymer composite as hereinbefore defined is obtained by known means.

Release of fluid may be in manner to foam the polymer substrate and create a porous structure, with deposition matter distributed throughout the polymer matrix and internal pore surface. Typically this is achieved by rapid release over a period of up to 2 minutes.

Depressurisation period may be selected to foam the polymer if desired, and therefore determines the porosity of composite. Transition is preferably rapid over a period of from 1 ms to 10 minutes, preferably from 1 second to 3 minutes, more preferably from 1 to 3 seconds for high porosity polymer. Alternatively plasticising fluid may be released in manner to allow fluid diffusion out of the polymer, avoiding foaming, to create a non-porous structure. Typically this is achieved by prolonged gradual release of fluid over a period of in excess of 10 minutes up to 12 hours. Preferably transition is to near ambient pressure i.e. substantially 1 atm (101.325 kPa).

The process may be carried out in the presence or absence of additional solvents or fluids. In the case of physical interaction of deposition matter with the polymer surface additional solvents or fluids may be used without affecting the uniform deposition layer. Preferably however the process is carried out in the absence of solvent capable of dissolving the deposition matter. Suitable carriers, agents, preservation agents and the like may be employed as desired.

A plasticising fluid as hereinbefore defined may comprise any fluid which is capable of plasticising a desired polymer. As is known in the art such fluids may be subjected to conditions of elevated temperature and pressure increasing density thereof up to and beyond a critical point at which the equilibrium line between liquid and vapour regions disappears. Supercritical and dense phase fluids are characterised by properties which are both gas like and liquid like. In particular, the fluid density and solubility properties resemble those of liquids, whilst the viscosity, surface tension and fluid diffusion rate in any medium resemble those of a gas, giving gas like penetration of the medium.

Preferred plasticising fluids include carbon dioxide, di-nitrogen oxide, carbon disulphide, aliphatic C₂₋₁₀ hydrocarbons such as ethane, propane, butane, pentane, hexane, ethylene, and halogenated derivatives thereof such as for example carbon tetrafluoride or chloride and carbon monochloride trifluoride, and fluoroform or chloroform, C₆₋₁₀ aromatics such as benzene, toluene and xylene, C₁₋₃ alcohols such as methanol and ethanol, sulphur halides such as sulphur hexafluoride, ammonia, xenon, krypton and the like, and mixtures thereof. Typically these fluids may be brought into plasticising conditions at temperature of between −200° C. to +500° C. and pressures of in excess of 1 bar to 10000 bar, as hereinbefore defined. It will be appreciated that the choice of fluid may be made according to its properties, for example diffusion and polymer plasticisation. Preferably the fluid acts as solvent for residual components of a polymer composite as hereinbefore defined but not for polymer or deposition matter as hereinbefore defined. Choice of fluid may also be made with regard to critical conditions which facilitate the commercial preparation of the polymer as hereinbefore defined. Supercritical conditions are shown of some fluids in Table 1.

| Fluid | Critical Temperature/° C. | Critical Pressure/bar |
|---|---|---|
| Carbon dioxide | 31.1 | 73.8 |
| Ethane | 32.4 | 48.1 |
| Ethylene | 9.3 | 49.7 |
| Nitrous oxide | 36.6 | 71.4 |
| Xenon | 16.7 | 57.6 |
| Fluoroform CHF₃ | 26.3 | 48.0 |
| Monofluoromethane | 42 | 55.3 |
| Tetrafluoroethane | 55 | 40.6 |
| Sulphur hexafluoride | 45.7 | 37.1 |
| Chlorofluoromethane | 29 | 38.2 |
| Chlorotrifluoromethane | 28.9 | 38.7 |
| Nitrogen | −147 | 33.9 |
| Ammonia | 132.5 | 111.3 |
| Cyclohexane | 280.3 | 40.2 |
| Benzene | 289.0 | 48.3 |
| Toluene | 318.6 | 40.6 |
| Trichlorofluoromethane | 198.1 | 43.5 |
| Propane | 96.7 | 41.9 |
| Propylene | 91.9 | 45.6 |
| Isopropanol | 235.2 | 47.0 |
| p-xylene | 343.1 | 34.7 |

Preferably the plasticising fluid comprises carbon dioxide optionally in admixture with any further fluids as hereinbefore defined or mixed with conventional solvents, so-called “modifiers”. CO₂ is generally approved by regulatory bodies for medical applications, is chemically inert, leaves no residue and is freely available.

The plasticising fluid may be present in any effective amount with respect to the polymer. Preferably the plasticising fluid is provided at a desired concentration in the reaction vessel to give a desired plasticisation and/or swelling of polymer. Such range may be from 1% to 200% of the polymer weight, e.g. with plasticising fluid in sufficient excess to achieve 10% to 40% absorption with respect to polymer weight.

The deposition matter may be present in any effective amount with respect to polymer. Typical values are therefore 1×10⁻¹² wt % to 99.9 wt %, preferably 0.01 or 0.1 to 99.0 wt %, more preferably greater than 0.5 wt % or 1.0 wt % up to 50 wt %. In a particularly preferred embodiment therefore the process is carried out in low volumes of the order of picogram and nanogram levels with respect to 5 g amounts of polymer. For example, presented as concentration of deposition matter on polymer, low volumes in the range 1×10¹ to 1×10³ ng/mg may be present, for example 50 to 150 ng/mg. This is beneficial for most biologically active molecules such as enzymes or protein molecules because their therapeutic concentrations are very low. For example: the therapeutic amount of the growth factor HGF (hepatocyte growth factor) required to provide a therapeutic response in liver cells during liver regeneration process in tissue engineering is 10 ng/ml ((Tsubouchi, Niitani et al. 1991).

The deposition matter may be selected from any desired matter adapted to perform a function on a desired biolocus comprising or otherwise associated with living matter, and which may be bioactive, bioinert, biocidal or the like; and non-biofunctional material including dyes, additives and the like.

Preferably deposition matter is selected from a component, or precursor, derivative or analogue thereof, of a host structure into which implantation or incorporation is desired and preferably comprises matter intended for growth or repair, shielding, protection, modification or modelling of a human, animal, plant or other living host structure for example the skeleton, organs, dental structure and the like; to combat antagonists; for metabolism of poisons, toxins, waste and the like or for synthesis of useful products by natural processes, for bioremediation, biosynthesis, biocatalysis or the like.

More specifically the deposition material includes but is not limited to the following examples typically classed as (pharmaceutical) drugs and veterinary products; agrochemicals as pest and plant growth control agents; human and animal health products; human and animal growth promoting, structural, or cosmetic products including products intended for growth or repair or modelling of the skeleton, organs, dental structure and the like; absorbent biodeposition materials for poisons, toxins and the like.

Pharmaceuticals and veterinary products, i.e. drugs, may be defined as any pharmacologically active compounds that alter physiological processes with the aim of treating, preventing, curing, mitigating or diagnosing a disease.

Drugs may be composed of inorganic or organic molecules, peptides, proteins, enzymes, oligosaccharides, carbohydrates, nucleic acids and the like.

Drugs may include but not be limited to compounds acting to treat the following:

Infections such as antiviral drugs, antibacterial drugs, antifungal drugs, antiprotozal drugs, anthelmintics,

Cardiovascular system such as positive inotropic drugs, diuretics, anti-arrhythmic drugs, beta-adrenoceptor blocking drugs, calcium channel blockers, sympathomimetics, anticoagulants, antiplatelet drugs, fibrinolytic drugs, lipid-lowering drugs;

Gastro-intestinal system agents such as antacids, antispasmodics, ulcer-healing, drugs, anti-diarrhoeal drugs, laxatives, central nervous system, hypnotics and anxiolytics, antipsychotics, antidepressants, central nervous system stimulants, appetite suppressants, drugs used to treat nausea and vomiting, analgesics, antiepileptics, drugs used in parkinsonism, drugs used in substance dependence;

Malignant disease and immunosuppresion agents such as cytotoxic drugs, immune response modulators, sex hormones and antagonists of malignant diseases;

Respiratory system agents such as bronchodilators, corticosteroids, cromoglycate and related therapy, antihistamines, respiratory stimulants, pulmonary surfactants, systemic nasal decongestants;

Musculoskeletal and joint diseases agents such as drugs used in rheumatic diseases, drugs used in neuromuscular disorders; and

Immunological Products and Vaccines.

Agrochemicals and crop protection products may be defined as any pest or plant growth control agents, plant disease control agents, soil improvement agents and the like. For example pest growth control agents include insecticides, miticides, rodenticides, molluscicides, slugicides, vermicides (nematodes, anthelmintics), soil fumigants, pest repellants and attractants such as pheromones etc, chemical warfare agents, and biological control agents such as microorganisms, predators and natural products;

plant growth control agents include herbicides, weedicides, defoliants, dessicants, fruit drop and set controllers, rooting compounds, sprouting inhibitors, growth stimulants and retardants, moss and lichen controllers and plant genetic controllers or agents;

plant disease control agents include fungicides, viricides, timber preservatives and bactericides; and

soil improvement agents include fertilisers, trace metal additives, bacterial action control stimulants and soil consolidation agents.

The deposition matter may alternatively or additionally comprise any function enhancing components, including naturally occurring or synthetic otherwise modified growth promoters, biocompatibilisers, vitamins, proteins, glycoproteins, enzymes, nucleic acid, carbohydrates, minerals, nutrients, steroids, ceramics and the like and functioning matter such as spores, viruses, mammalian, plant and bacterial cells. Preferred deposition matter includes growth factors selected from biocompatibilisers, vitamins, proteins, glycoproteins, enzymes, nucleic acid, carbohydrates, minerals, nutrients, steroids, ceramics and the like; in particular growth factors such as basic Fibroblastic Growth Factor, acid Fibroblastic Growth Factor, Epidermal Growth Factor, Human Growth Factor, Insulin Like Growth Factor, Platelet Derived Growth Factor, Nerve Growth Factor and Transforming Growth Factor and bone morphogenetic proteins; antitumorals such as BCNU or 1,3-bis (2-chloroethyl)-1-nitrosourea, daunorubicin, doxorubicin, epirubicin, idarubicin, 4-demethoxydaunorubicin 3′-desamine-3′-(3-cyano-4-morpholinyl)-doxorubicin, 4-demethoxydaunorubicin-3′-desamine-3′-(2-methoxy-4-morpholinyl)-doxorubicin, etoposide and teniposide; hormones such as LHRH and LHRH analogues; and steroideals for birth control and/or antitumoral action such as medroxyprogesterone acetate or megestrol acetate; tricalcium phosphate or the class of apatite derivatives, for example calcium hydroxyapatite which functions as a bone or dental component and promotes biocompatability, silicon which functions as a tissue modelling component, and analogues, precursors or functional derivatives thereof, bioactive species such as collagen, bioglasses and bioceramics, other minerals, hyaluran, polyethyleneoxide, CMC (carboxymethylcellulose), proteins, organic polymers, and the like and components adapted for incorporation as implants into meniscus, cartilage, tissue and the like and preferably promote growth, modelling, enhancing or reinforcing of collagen, fibroblasts and other natural components of these host structures.

Absorbent deposition matter for poisons, toxins and the like may be defined as any natural or synthetic products capable of immobilising by absorption, interaction, reaction or otherwise of naturally occurring or artificially introduced poisons or toxins.

The deposition matter may be in any desired form suited for the function to be performed, for example in solid, semi-solid such as thixotrope or gel form, semi-fluid or fluid such as paste or liquid form, and may be miscible or immiscible but is insoluble in the polymer and plasticising fluid, eg as a suspension. It may be convenient to adapt the deposition matter form to render it in preferred form for processing and the function to be performed. The matter is preferably in the form of solid particles having particle size selected according to the desired application. Preferably particle size is of similar or of lesser order to that of the polymer composite, and optionally of any pores, preferably 10⁻⁹ m-10⁻² m, for example of the order of picometers, nanometers, micrometers, millimetres or centimetres.

The polymer composite may be in desired form suitable for the hereinbefore mentioned uses. For application to living matter, the polymer composite may be introduced as a dry or wet spray, powder, pellets, granules, monoliths and the like, comprising the deposition material substrate in releasable manner by dissolution, evaporation or the like, for example in the hereinbefore defined agrochemical, insecticidal and the like uses. For administration as a healthcare, pharmaceutical or the like composition to the human or animal body, the composition may be suitably formulated according to conventional practices.

For use as pharmaceutical and veterinary products fabricated using the inventive process composites may be in the form of creams, gels, syrups, pastes, sprays, solutions, suspensions, powders, microparticles, granules, pills, capsules, tablets, pellets, suppositories, pessaries, colloidal matrices, monoliths and boluses and the like, for administration by topical, oral, rectal, parenteral, epicutaneous, mucosal, intravenous, intramuscular, intrarespiratory or like.

The composite may be non porous or porous, and may comprise open or closed cell pores. Composite obtained with a very open porous structure, known as microcellular, is ideal for prolonged or staged release, for pharmaceutical and animal health etc applications as hereinbefore defined, also for biomedical and biocatalytic applications for example supporting growth of blood vessels and collagen fibres throughout the matrix, and forming structures resembling bone, meniscus, cartilage, tissue and the like, and providing a structure for throughput of substrate for biocatalysis and bioremediation and the like.

Non-porous, open or closed cell composite may be useful for biodegradable staged or prolonged release delivery applications of deposition matter not requiring leaching in or out or other access. Release may be in vitro or in vivo and by parenteral, oral, intravenous, application or surgical for release proximal to the treatment locus, eg in tissue tumor treatment, or hyperthermic bone tumor treatment.

A porous polymer composite may be obtained with uniform or varied porosity, preferably provides pores of at least two different orders of magnitude, for example of micro and macro type, each present in an amount of between 1 and 99% of the total void fraction of the polymer composite.

Reference herein to micro and macro pores is therefore to be understood to be respectively pores of any unit dimension and its corresponding 10ⁿ multiple. For example micro pores may be of the order of 10 ⁻⁽¹⁰⁻⁷⁾ m with respective macro pores of the order of 10 ⁻⁽⁷⁻⁵⁾ m, preferably 10 ⁻⁽⁸⁻⁷⁾ m and 10 ⁻⁽⁶⁻⁵⁾ m respectively, more preferably of micron and 10² micron order, for example 50 to 200 micron. The pores may be of any desired configuration. Preferably the pores form a network of tortuous interlinking channels, more preferably wherein the micro pores interlink between the macro pores.

Deposition matter may be distributed throughout relatively smaller and relatively larger pores or confined to larger pores. Deposition matter may be embedded in the walls of pores or may be freely supported but not encased in polymer matrix.

An open cell structure may create a channel structure throughout the polymer composite, for leaching in and out of fluids for prolonged release, or for supply and removal of materials, in particular fluids and release matter. Different particle size deposition matter may selectively distribute between smaller and larger pores.

A composite created in this manner may enhance the biomechanical properties of the polymer, in contrast to that of known polymers comprising inhomogeneous distribution and large aggregates of inorganic materials.

The process may be controlled in manner to determine the dimensions and void fraction of micro and macro pores and the morphology of the final product. The period for plasticising fluid release determines in part the level of porosity. Additionally the difference in pressure is proportional to porosity. Also a higher critical temperature confers a higher porosity. The composite is suitably obtained with porosity of 15% to 75% or greater, preferably 50% up to 97%.

Suitably the polymer retains its solid or highly viscous fluid form subsequent to release of plasticising fluid, in order to retain the porous structure induced by the fluid.

Further processing of the polymer, for example additional extraction with super critical fluid as known in the art or with other extractants, post-polymerisation and cross-linking, may be subsequently performed as required and as known in the art.

The polymer may be selected from any known polymer, (block) copolymer, mixtures and blends thereof which may be crosslinked or otherwise, which is suited for introduction into or association with the human or animal body, plants or other living matter, or in vitro, or for use in the environment in non-toxic manner. Suitable polymer materials are selected from synthetic biodegradable polymers as disclosed in “Polymeric Biomaterials” ed. Severian Dumitriu, ISBN 0-8247-8969-5, Publ. Marcel Dekker, New York, USA, 1994, bioresorbable polymers synthetic non-biodegradable polymers; and natural polymers. Preferably the polymer is selected from homopolymers, block and random copolymers, polymeric blends and composites of monomers which may be straight chain, (hyper) branched or cross-linked.

Polymer may be of any molecular weight for the desired application, and is suitably in the range of from 1 to 1,000,000 repeat units. Higher molecular weight may be useful for longer release patterns or slower degradation.

Polymers may include but are not limited to the following which are given as illustration only.

Synthetic biodegradable polymers may be selected from:

Polyesters including poly(lactic acid), poly(glycolic acid), copolymers of lactic and glycolic acid, copolymers of lactic and glycolic acid with poly(ethylene glycol), poly(e-caprolactone), poly(3-hydroxybutyrate), poly(p-dioxanone), poly(propylene fumarate);

Preferably polylactides include DD, DL, LL enantiomers and are prepared from D and L lactic acid and glycolic acid monomers, or a combination thereof, or monomers such as 3-propiolactone tetramethylglycolide, b-butyrolactone, 4-butyrolactone, pivavolactone and intermolecular cyclic esters of alpha-hydroxy butyric acid, alpha-hydroxyisobutyric acid, alpha-hydroxyvaleric acid, alpha-hydroxyisovaleric acid, alpha-hydroxycaproic acid, alpha-hydroxy-alpha-ethylbutyric acid, alpha-hydroxyisocaproic acid, alpha-hydroxy-3-methylvaleric acid, alpha-hydroxyheptanoic acid, alpha-hydroxyoctanoic acid, alpha-hydroxydecanoic acid, alpha-hydroxymyristic acid, alpha-hydroxystearic acid, and alpha-hydroxylignoceric acid. It is most preferred to use lactic acid as sole monomer or lactic acid as the principal monomer with glycolic acid as the comonomer. The latter are termed poly(lactide-co-glycolide) copolymers; particularly suitable are polymers prepared from lactic acid alone, glycolic acid alone, or lactic acid and glycolic acid wherein the glycolic acid is present as a comonomer in a molar ratio of 100:0 to 40:60;

Poly (ortho esters) including Polyol/diketene acetals addition polymers as described by Heller in: ACS Symposium Series 567, 292-305, 1994;

Polyanhydrides including poly(sebacic anhydride) (PSA), poly(carboxybisbarboxyphenoxyphenoxyhexane) (PCPP), poly[bis(p-carboxyphenoxy) methane] (PCPM), copolymers of SA, CPP and CPM, as described by Tamada and Langer in Journal of Biomaterials Science—Polymer Edition, 3, 315-353,1992 and by Domb in Chapter 8 of the Handbook of Biodegradable Polymers, ed. Domb A. J. and Wiseman R. M., Harwood Academic Publishers;

Poly(amino acids); polyacetals; polyketals; polyorthoesters;

Poly(pseudo amino acids) including those described by James and Kohn in pages 389-403 of Controlled Drug Delivery Challenges and Strategies, American Chemical Society, Washington D.C.;

Polyphosphazenes including derivatives of poly[(dichloro) phosphazene], poly[(organo) phosphazenes], polymers described by Schacht in Biotechnology and Bioengineering, 52, 102-108, 1996; and

Azo Polymers

Including those described by Lloyd in International Journal of Pharmaceutics, 106, 255-260, 1994.

Synthetic Non-biodegradable Polymers may be selected from:

Vinyl polymers including polyethylene, poly(ethylene-co-vinyl acetate), polypropylene, poly(vinyl chloride), poly(vinyl acetate), poly(vinyl alcohol) and copolymers of vinyl alcohol and vinyl acetate, poly(acrylic acid) poly(methacrylic acid), polyacrylamides, polymethacrylamides, polyacrylates, Poly(ethylene glycol), Poly(dimethyl siloxane), Polyurethanes, Polycarbonates, Polystyrene and derivatives.

Natural Polymers may be selected from carbohydrates, polypeptides and proteins including:

Starch, Cellulose and derivatives including ethylcellulose, methylcellulose, ethylhydroxyethylcellulose, sodium carboxymethylcellulose; Collagen; Gelatin; Dextran and derivatives; Alginates; Chitin; and Chitosan;

Preferably a non biodegradable polymer is selected from polymers such as ester urethanes or epoxy, bis-maleimides, methacrylates such as methyl or glycidyl methacrylate, tri-methylene carbonate, di-methylene tri-methylene carbonate; biodegradable synthetic polymers such as glycolic acid, glycolide, lactic acid, lactide, p-dioxanone, dioxepanone, alkylene oxalates and caprolactones such as gamma-caprolactone.

Polymer substrate may be obtained from its precursors in the process of the invention. The precursors may react to form the polymer substrate(s) in situ during or subsequent to plasticising fluid processing.

The polymer may comprise any additional polymeric components having performance enhancing or controlling effect, for example determining the degree and nature of cross-linking for desired degradation, release, or fluid access, flexural and general mechanical properties, electrical properties and the like.

Additional components which may be incorporated during the manufacture of the polymer composite, for example other active agents, initiators, accelerators, hardeners, stabilisers, antioxidants, adhesion promoters, fillers and the like may be incorporated within the polymer. Additional materials(s) may be mixed with the polymer before or after contacting with deposition matter, or may be introduced by subsequent soaking or impregnation of the product composite having internally distributed deposition matter.

If it is desired to introduce an adhesion promoter into the polymer composite, the promoter may be used to impregnate or coat particles of deposition matter prior to introduction into the polymer composite, by means of simple mixing, spraying or other known coating steps, in the presence or absence of fluid as hereinbefore defined. Preferably coating is performed in conjunction with mixing with fluid as hereinbefore defined whereby excellent coating is obtained. For example the adhesion promoter is dissolved in fluid as hereinbefore defined and the solution is contacted with particles of deposition matter as hereinbefore defined. Alternatively the adhesion promoter is introduced into the autoclave during the mixing and/or polymerisation step whereby it attaches to particles of deposition matter in desired manner.

Preferably the total amount of fillers including the deposition matter lies in the region of 0.01-99.9 wt %, preferably 0.1-99 wt %, more preferably in excess of 50 or 60 wt %, up to for example 70 or 80 wt %.

In some cases it may be desirable to introduce an initiator or accelerator to initiate (partial) curing prior to and/or subsequent to release of fluid, and initiation may be simultaneous with introduction or may be delayed, activated by increase in temperature. Alternatively a spray drying step may be employed in place of the curing step prior to or simultaneously with release of the fluid. In this case a post-curing may be employed. This may have advantages in terms of ease of manufacturing and simplicity of apparatus employed.

In a further aspect of the invention there is provided a polymer composite obtained with the process of the invention as hereinbefore defined.

In a further aspect of the invention there is provided a polymer composite comprising a porous or non porous polymer throughout which particulate deposition matter as hereinbefore defined is distributed with desired uniformity, preferably with high uniformity in excess of 80% for example in excess of 98%. In a particular advantage the composite comprises exceedingly low levels of deposition matter of the order of picograms or nanograms per 5 g polymer, or presented as concentration of deposition matter on polymer, in low volumes in the range 1×10¹ to 1×10³ ng/mg at excellent levels of uniformity and batch reproducibility, and/or of very low particle size of the order of 10 microns, 1 micron or 0.1 microns.

In a further advantage, contrasted with other methods of encapsulating (e.g. double emulsion) and introducing biological material which give rise to relatively large particles which give an uneven release with time, the process of the present invention enables internally distributing very small particles of deposition matter thus giving a much even release profile (reduced burst phase effect). Moreover the composite of the invention has been found to give release over a period of several months, and this is in contrast to the corresponding surface deposited polymer which may lose its surface deposit over the course of days.

The composite of the invention may be distinguished from prior art composite prepared by simple impregnation techniques and those of WO 91/09079 which show agglomeration of impregnation matter etc.

Advantageously it has been found that very low and very high loading may be obtained according to the process of the present invention, which is not possible with known processes, by virtue of the uniform morphology of polymer and deposition matter, and loadings of deposition matter in the range from 1×10⁻¹²-99.9 wt %, for example in the region 1×10⁻¹² to 1×10⁻⁹ wt %, midrange of from 20 to 50 wt % or in excess of 50 wt %, or in excess of 80 wt % may be obtained.

The polymer composite may be in desired form suitable for the hereinbefore mentioned uses. Suitably the composite may be obtained in granular or monolith form and is preferably in monolith form for use as a scaffold or drug delivery device.

For use as bioremediation, biocatalyst or biobarrier for human or animal or plant matter, the composite may be in a suitable shaped form or may be impregnated into a shaped product, to provide a barrier film, membrane, layer, clothing or sheet.

For use as a structural component, for example comprising the polymer and optional additional synthetic or natural metal, plastic, carbon or glass fibre mesh, scrim, rod or like reinforcing for medical or surgical insertion, the composite may be adapted for dry or wet insertion into a desired host structure, for example may be in powder, pellet, granule or monolith form suited for insertion as a solid monolith into bone or tissue, as fillers or cements for wet insertion into bone or teeth or as solid aggregates or monoliths for orthopaedic implants such as pins, or dental implants such as crowns etc. Insertion may be by injection, surgical insertion and the like.

The polymer composite may be of any desired particle size in the range of 0.1 or 1 micron powders, preferably from 50 or 200 micron for use with larger particle size deposition matter up to monoliths of the order of 20 centimetres magnitude. It is a particular advantage of the present invention that the polymer composite is obtained in the desired form in uniform size particles such as powder, pellets and the like. Accordingly if it is desired to obtain a random or discrete distribution of particle size the polymer composite may be milled or may be blended from different size batches.

Composite particle size may be controlled according to known techniques by controlled removal of plasticising fluid. If it is desired to obtain particulate composite, the process mixture is suitably removed from the mixing chamber under plasticising conditions into a separate container under ambient conditions through a nozzle or like orifice of desired aperture, and under desired difference of conditions and removal rate, adapted to provide the desired particle size. Spray drying apparatus and techniques may commonly be employed for the technique.

If it is desired to obtain a polymer composite in the form of monoliths, the plasticising fluid is suitably removed using known techniques for foaming polymers. Accordingly the polymer mix is retained in the reaction vessel and conditions are changed from plasticising to ambient at a desired rate to cause removal of the fluid from the polymer mix. Depending on the nature of the polymer it is possible to obtain the monolith in porous foamed state if desired, having interconnecting pores and channels created by the removal of the plasticising fluid, simply by selecting a polymer consistency which is adapted to retain its foamed state.

Monoliths may be formed into desired shape during the processing thereof, for example by removal of plasticising fluid from a mixing vessel, or from a mould internal to mixing vessel having the desired monolith shape. Alternatively monolith may be removed from the mixing vessel and cut to desired shape or transferred directly into a mould.

In a further aspect of the invention there is provided a scaffold comprising a polymer composite having internally distributed deposition matter as hereinbefore defined, suitably sized and shaped for a desired application as hereinbefore defined.

A scaffold according to the invention is suitably in the form of a surgical implant, synthetic bone composite, organ module, biocatalyst for remediation or synthesis, or the like. The scaffold may be biodegradable or otherwise, for biodegradation in the body and ingrowth by native cells, or for biodegradation in the environment after completion of bioremediation avoiding in each case the need for subsequent operation to remove the polymer.

In a further aspect of the invention there is provided an apparatus for use in the preparation of a polymer composite as hereinbefore defined. Suitably the apparatus comprises one or more pressure vessels adapted for temperature and pressure elevation and comprising means for mixing the contents. The pressure vessel may include means for depressurisation or for discharging of contents into a second pressure vessel at lower pressure. The apparatus comprises means for introduction of polymer, deposition matter and plasticising fluid and any other materials whilst the vessel is pressurised, as commonly known in the art.

In a further aspect of the invention there is provided a polymer composite as hereinbefore defined or a scaffold thereof for use as a support or scaffold for drug delivery, for use in bioremediation, as a biocatalyst or biobarrier for human or animal or plant matter, for use as a structural component, for example comprising the polymer and optional additional synthetic or natural metal, plastic, carbon or glass fibre mesh, scrim, rod or like reinforcing for medical or surgical insertion, for insertion as a solid monolith into bone or tissue, as fillers or cements for wet insertion into bone or teeth or as solid aggregates or monoliths for orthopaedic implants such as pins, or dental implants such as crowns etc.

The invention is now illustrated in non limiting manner with reference to the following examples and Figures wherein.

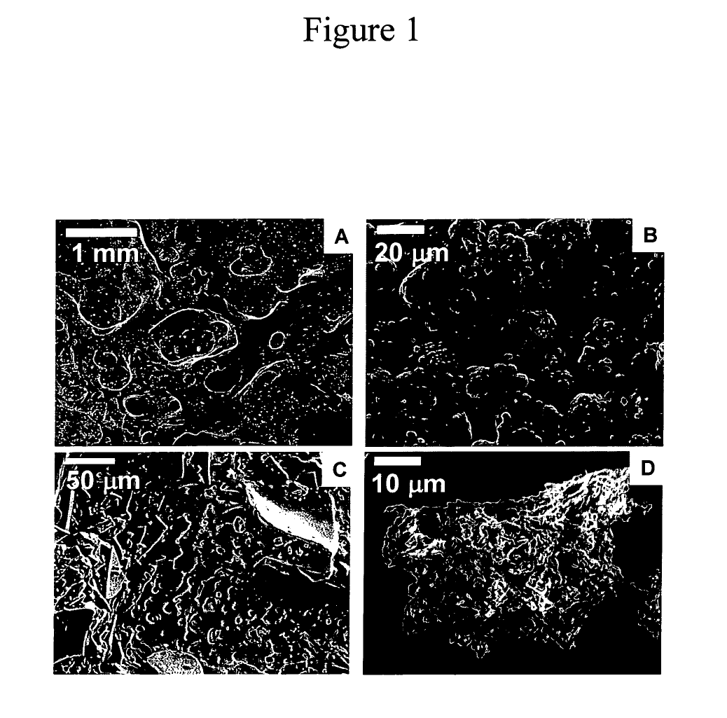

FIG. 1A-D shows scanning electron micrograph images of composites fabricated by the process of WO 98/51347 (Howdle et al) employed in the present invention; in Images A and B of an internal fracture surface of a monolith composite of calcium hydroxyapatite (40 wt %) and PLGA (60 wt %), at low magnification the distribution of calcium hydroxyapatite throughout the matrix and the production of pores is evident, at higher magnification the intimate mixing of guest particles and polymer is observed; in image C catalase (50% wt) is shown incorporated into a PLGA matrix (50%), micron scale pores in the polymer and the distinctive protein particle morphology are evident; in image D a high surface area microparticle composite (fluorescein (sodium salt) (8 wt %) and polycaprolactone (92 wt %)) are observed produced by direct atomisation, ie after fast depressurisation through an orifice.

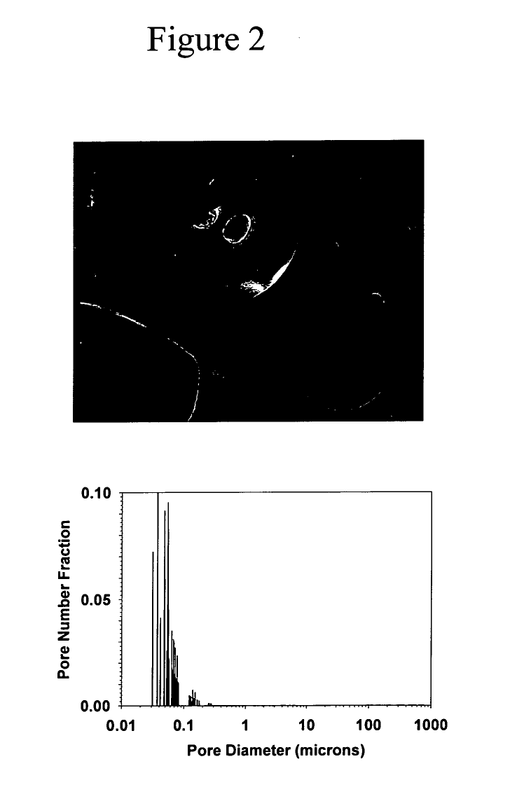

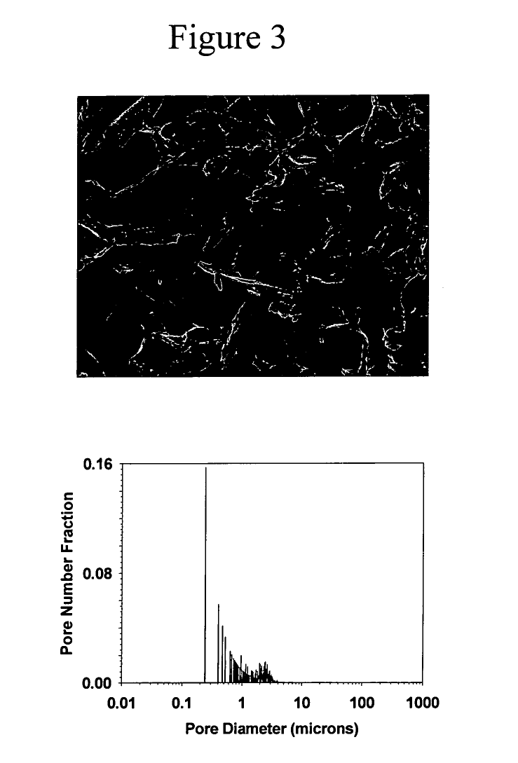

FIGS. 2 and 3 show scanning electron micrograph images and corresponding mercury porosimetry data for PLA composites fabricated by the process of WO 98/51347 (Howdle et al) employed in the present invention with control of PLA pore structure by changing de-pressurisation conditions; in FIG. 2 the image shows presence of a small population of large pores obtained by de-pressurisation over a 2-hour period (“slow”); in FIG. 3 the image shows an increase in porosity and a more heterogeneous distribution obtained by de-pressurisation over a 2-minute period (“fast”); data obtained by mercury porosimetry demonstrate that fine control over micropore distribution is achieved by changing only the de-pressurisation rate, with “slow” depressurisation creating pores in the 50 to 500 nm range, whilst “fast” depressurisation is strikingly different and creates pores in the 500 nm to 5 μm range.

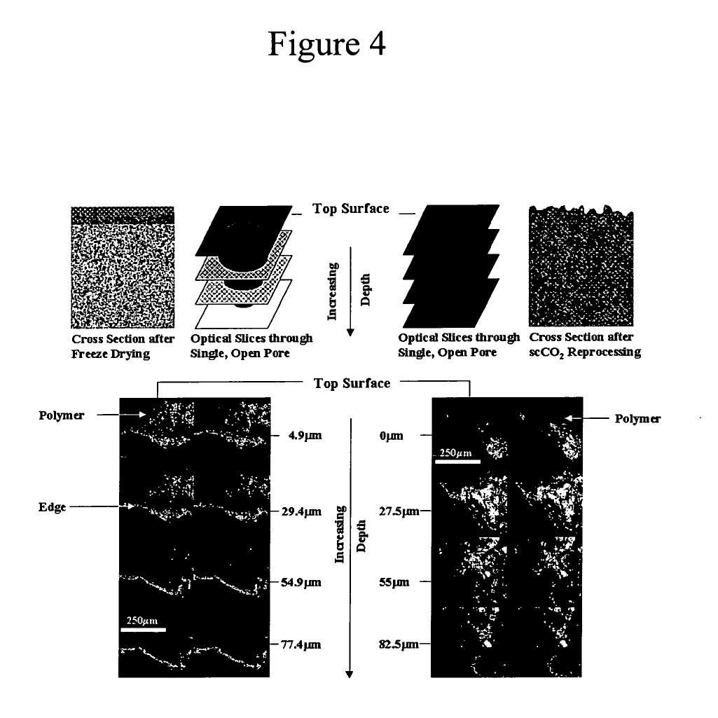

FIG. 4 shows a schematic of the method of the invention in which fluorescent protein solution is adsorbed onto the polymer surface, the protein is confined to the surface and does not penetrate the bulk; confocal cross section through the polymer from the top surface shows protein confined to the edge and outer pores of the PLA scaffold; thereafter the polymer: protein complex is plasticised in CO₂, the protein is shown distributed throughout the sample, and the resulting fluorescence is homogeneous with the protein redistributed from the surface to the bulk of the polymer.

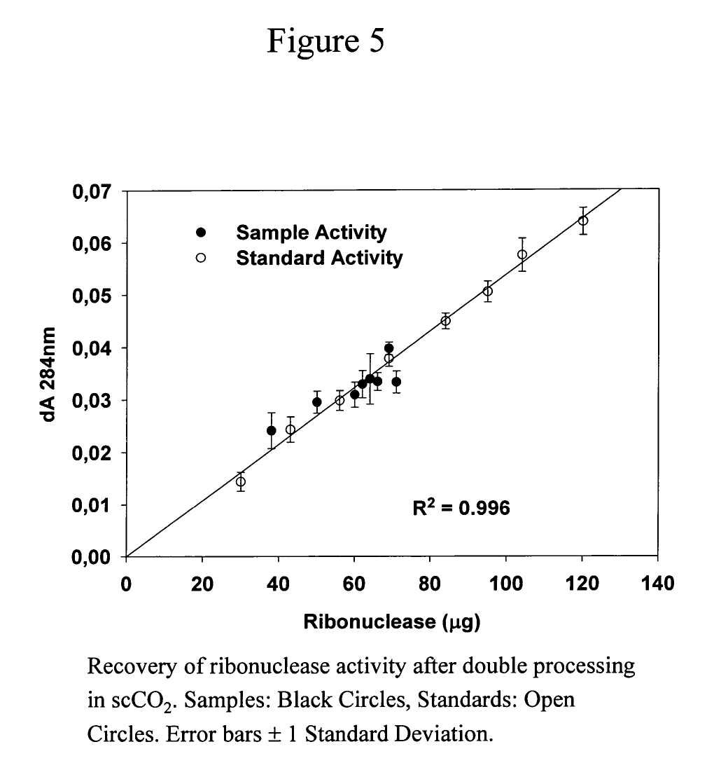

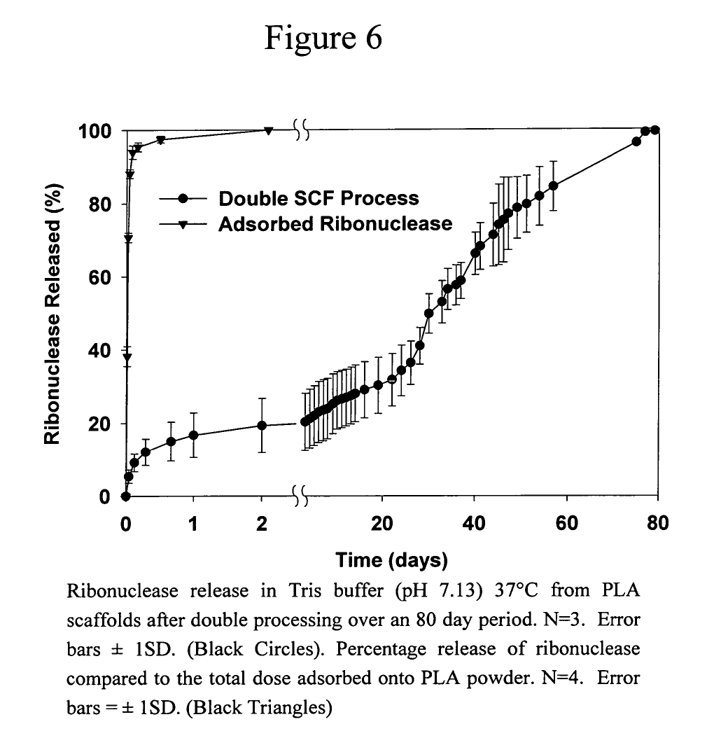

FIG. 5 shows recovery of protein activity after double processing in CO₂ FIG. 6 shows protein release with time for the composite of FIG. 4 and comparative composite not according to the invention.

### METHODS AND MATERIALS

Cell Culture

Bone marrow samples (16 patients in total: 11 females and 5 males aged 14-83, with a mean age of 63.8 years) were obtained from patients undergoing routine total hip replacement surgery. Only tissue, which would have been discarded, was used with ethical approval. Human bone marrow cells were cultured on poly(lactic acid) porous scaffolds encapsulated with and without recombinant human BMP-2 or PLA scaffolds adsorbed with rhBMP-2. In vitro assays included human bone marrow cells with or without addition of recombinant human BMP-2 (50 ng/ml) in basal (10% αMEM) and osteogenic conditions (10% αMEM supplemented with 100 μg/ml ascorbate and 10 nM dexamethasone).

Chorioallantoic Membrane Assay

Fertilised eggs were incubated for 10-18 days using a Multihatch automatic incubator (Brinsea Products, Sandford, UK) at 37° C. in a humidified atmosphere. Chick femurs were excised from day 18 chick embryos and a wedge-shaped segmental defect created in the middle of the femur, into which the scaffold construct was placed to fill the defect site. Chick bone and scaffolds (29 samples) were placed directly onto the CAM of 10-day-old eggs (through a 1 cm² square section cut into the shell) and incubation continued for a further 7 days. The femoral/scaffold explant was then placed onto the CAM and incubation, at 37° C., continued for a further 7 days. Explants were then harvested and the chick embryo killed by decapitation. Prior to histochemical analysis, scaffold and explant samples were then fixed in 95% ethanol, processed to paraffin wax and 5 μm sections prepared for histology.

### EXAMPLE 1

### Preparation of Polymer Material

Poly(DL-lactic acid) (Alkermes Medisorb, low I.V. Mw=85 kD, polydispersity=1.4) was ground to a fine grain size powder in a pestle and mortar. Alternatively, particles were produced by forcing the poly(DL-lactic acid) out of a vessel pressurized with CO₂ through an orifice. The particles were retrieved from a cyclone collector, the CO₂ may be repressurised and recycled. The methodology is based on the antisolvent technique of particle generation from supercritical suspension (PGSS).

The polymer may also be prepared as a highly porous monolith using supercritical fluid processing. In this case porous scaffolds were prepared in moulds prepared from 48-well tissue culture plates (Costar, USA). 12×100 mg (+1 mg) PLA were weighed out into the wells, and the mould was sealed inside the autoclave. The autoclave was heated to 35° C. before filling with CO₂ over a period of 30 minutes to a pressure of 207 Bar. This long filling time minimised the potentially detrimental effects of excessive Joule-Thompson heating on the biologically active substrate as the system was pressurised. The plasticising CO₂-polymer mixture was allowed to equilibrate for 20 minutes before venting to atmospheric pressure over 8 minutes. The pressure was controlled throughout the preparation using a JASCO BP-1580-81 programmable backpressure regulator. The autoclave temperature remained below 38° C. throughout the filling step, and the flow rate of CO₂ during the equilibration step was 12 cm³ min⁻¹. After the CO₂ processing, the mould containing the foamed polymer was removed from the autoclave and the residual gas allowed to escape for 2 hours.

### EXAMPLE 2

### Addition of the Biological Material—Protein

The protein, in this example avidin tagged with the fluorescent molecule rhodamine (Sigma), was dissolved in distilled water to give solutions at a concentration of 1 microgram and 10 microgram per ml in water). The liquid may alternatively be chosen from any liquid that dissolves the biological molecule but does not dissolve the polymer. 0.5 cm³ aliquots of protein solution were pipetted onto approx 250 mg samples of polymer material and remained in contact with the samples for a period of between 1 sec and 48 hours. During this exposure, a freeze drying process was used to remove the liquid. We have freeze-dried a range of avidin-rhodamine and ribonuclease solutions (1 microgram −250 mg/ml) onto both porous scaffolds and polymer powders for periods of up to 48 hours. Control scaffolds without any protein addition were prepared.

Confocal fluorescence microscopy of this material confirmed that the avidin rhodamine was confined to the surface of the polymer material and was not distributed with the solid mass of the polymer (FIG. 4).

### EXAMPLE 3

### Re-Distribution of the Biological Material—Protein

One scaffold from each protein concentration sample from Example 2, was removed from the well to act as control. The remaining examples were placed into a high pressure autoclave and heated to 35° C., replasticised in CO₂ using the same procedure as Example 2 above. FIG. 4 shows a schematic of the plasticising process. Confocal fluorescence microscopy of this re-processed material showed that the avidin rhodamine was re-distributed within the bulk of the polymer (FIG. 4). Confocal microscopy was performed using a Leica TCS4D system with a Leica DMRBE upright fluorescence microscope and an argon-krypton laser. The red fluorescence of TRITC Avidin-Rhodamine was excited with the 568 nm laser line.

### EXAMPLE 4

### Addition of Biological Material—Enzyme

To prove that the activity of biological material was unaffected by this treatment, 100 microlitres of 250 mg/ml of the enzyme ribonuclease A (Sigma) was adsorbed onto 8 batches of 100 mg poly(DL-lactic acid) powder using the method of the above Examples and freeze-dried for 48-hours.

### EXAMPLE 5

### Redistribution of Biological Material—Enzyme

The powder of Example 4 was processed using the conditions in Example 3 to produce polymer foam composites.

### EXAMPLE 6

### Evidence for Retention of Activity

The ribonuclease enzyme was released from the foams obtained in Example 5 in a Tris buffer (pH 7.13) at physiological temperatures. Using a specific ribonuclease substrate, cytidine-2′:3′-monophospate, the recovery of activity was monitored by the conversion of the substrate to a form that could be detected by a UV spectrophotometer (Table 1). Full biological activity of the protein was retained.

Results

FIG. 4 shows a schematic of the supercritical fluid process. Concentration profiles of the fluorescent avidin-rhodamine complex are shown after the freeze-drying step and after plasticising CO₂ reprocessing. Following the initial freeze-drying, fluorescence is localised at the exposed surfaces of the scaffold, i.e. the top surface and the walls of pores. After CO₂ reprocessing, the complex is distributed throughout the sample, and the resulting fluorescence is homogeneous.

The schematic is supported by data from confocal microscopy. On the left are eight images that follow the edge of a pore in a sample from the top surface to a depth of 77.4 μm after the initial freeze-drying step. The images show a decreasing intensity of fluorescence as the distance from the top surface increases, except for a narrow region localised at the edge of the pore.

The series on the right depicts a sample that has been reprocessed in plasticising CO₂. Here again, the series follows the edge of a pore to a depth of 82.5 μm below the surface. In contrast to the unprocessed scaffold, fluorescence is observed throughout the scaffold with appreciable intensity seen both in the bulk and at the pores' surface.

Ribonuclease activity was measured after release into Tris buffer solution from scaffolds after processing in scCO₂ (FIG. 5). The rate of reaction of conversion of cytidine-2′,3′-monophosphate to cytidine-3′-phosphate was measured by the change in absorbance at 284 nm. The black circles (samples) represent the activity of the enzyme compared to the standards (open circles). The mean recovery of activity was 100.8% (+9.8%) indicating that enzyme activity is

| | Actual | Maximal | | | Percentage |
|---|---|---|---|---|---|
| Sam- | Amount RN | Rate | Actual Rate | Standard | Recovery |
| ple | (microgram) | (dA 284 nm) | (dA 284 nm) | Deviation | (%) |
| 1 | 66 | 0.0354 | 0.0334 | 0.0017 | 94.4 |
| 2 | 69 | 0.0374 | 0.0397 | 0.0012 | 106.2 |
| 3 | 71 | 0.0384 | 0.0333 | 0.0024 | 86.8 |
| 4 | 60 | 0.0323 | 0.0309 | 0.0021 | 95.5 |
| 5 | 50 | 0.0270 | 0.0295 | 0.0021 | 109.4 |
| 6 | 64 | 0.0345 | 0.0339 | 0.0048 | 98.3 |
| 7 | 62 | 0.0334 | 0.0329 | 0.0026 | 98.4 |
| 8 | 38 | 0.0205 | 0.0241 | 0.0034 | 117.4 |

retained throughout the process. The correlation between sample and standard activity is high (R²=0.9959).

### EXAMPLE 7

### Evidence of Controlled Release

FIG. 6 displays the protein release behaviour from Example 6 as a function of time. Where the protein has been dried onto the polymer scaffold without a second plasticising CO₂ processing step, the protein is released very quickly with nothing remaining after two days (Black triangles). In samples which have been subjected to the SCF reprocessing step, the release is far more protracted. After an initial “burst” phase (0-1 days), the rate of release stabilises for approximately three weeks before degradation of the polymer matrix allows the protein to escape. The profile then follows a rectilinear relationship until the exhaustion of the protein after approximately 80 days.

### EXAMPLE 8

### Addition of Biological Material—Growth Factor

Scaffold Generation and rhBMP-2 Encapsulation

Polymer obtained as in Example 1 was loaded with the Growth Factor recombinant human bone morphogenetic protein-2 (rhBMP-2). Poly(DL-lactic acid) and rhBMP-2 (100 ng/mg PLA) were mixed together using a combination of conventional solution and supercritical carbon dioxide processing to generate porous (50-200 μm) scaffolds. Recombinant BMP-2 was adsorbed onto poly(D,L-lactic acid) powder (Alkermes Inc., USA; low inherent viscosity, Mw 84 kDa, polydispersity=1.4) at a concentration of 100 ng/mg polymer. The polymer:protein mixture was processed using a supercritical carbon dioxide pressurized to 207 bar and heated to 35° C. for 20 minutes in a high pressure vessel. Upon depressurization, the protein is encapsulated within the polymer and pores are formed in the polymer matrix by the escape of the carbon dioxide gas. Functionally active recombinant human BMP-2 was derived from E. Coli, at greater than 98% purity in a largely homogenous form. In this procedure, the efficient processing of the liquefied polymer in scCO₂ at near ambient temperatures results in a homogeneous distribution of the bioactive factor throughout the polymer matrix. These mild processing conditions allow the processing of growth factors that are heat or solvent sensitive without further degradation or damaging their biological activity.

### EXAMPLE 9

### Cell Growth in PLA

Human bone marrow cell/PLA constructs were cultured in 10% FCS αMEM supplemented with osteogenic medium containing 5 mM inorganic phosphate for the final 48 hours of the culture period and mineralization was detected by von Kossa staining.

Histochemistry and Immunocytochemistry

Prior to histochemical analysis, PLA scaffold samples were fixed with 4% Paraformaldehyde or 95% ethanol, dependent on the staining protocol and, as appropriate, processed to paraffin wax and 5 μm sections prepared. Negative controls were included in all studies. i) Alkaline phosphatase activity: Cultures stained using the Sigma alkaline phosphatase kit (no.85) according to the manufacturer's instructions; ii) Alcian blue/Sirius red: Samples were stained using Weigert's haematoxylin, 0.5% alcian blue (in 1% acetic acid) and sirius red (in saturated Picric acid). iii) Toluidine Blue and Von Kossa Staining: Samples were stained with 1% AgNO3 under UV light for 20 minutes until black deposits were visible and after air drying, slides were counterstained with toluidine blue.

C2C12 Alkaline Phosphatase Assay

BMP-2 has the ability to induce C2C12 promyoblast differentiation into the osteoblast lineage^{(33,34,35)}. After encapsulation of 0.01% (w/w) rhBMP-2 within PLA scaffolds, the bioactivity of rhBMP-2 released from the polymer was determined using C2C 12 cells. Briefly, human bone marrow stromal cells were cultured in the presence or absence of rhBMP-2 encapsulated PLA scaffold, or passaged onto rhBMP-2 encapsulated PLA scaffold or PLA scaffold alone in 10% FCS DMEM at 37° C. and 5% CO₂ for three days. Samples were fixed in ethanol and stained for alkaline phosphatase.

Bioactivity of rhBMP-2 Encapsulated PLA Scaffolds

After encapsulation of rhBMP-2 within PLA scaffolds (100 ng/mg PLA), the bioactivity of rhBMP-2 released from PLA scaffolds was determined using induction of the C2C 12 promyoblast cell line into the osteogenic lineage as detected by alkaline phosphatase expression. Alkaline phosphatase-positive cells were observed following culture of C2C12 cells in presence of or on rhBMP-2 encapsulated PLA scaffolds (FIGS. 1A, C). No induction of alkaline phosphatase-positive cells was observed using blank scaffolds (FIGS. 1B, D). As expected, rhBMP-2 (50 ng/ml) adsorbed on PLA promoted human bone marrow stromal cell adhesion, spreading, proliferation, and differentiation on PLA porous scaffold in vitro as observed by SEM, confocal microscopy and expression of type I collagen histochemistry (data not shown).

Human Osteoprogenitor Growth on rhBMP-2 Encapsulated Scaffolds

Following demonstration of the ability of using rhBMP-2 encapsulated PLA scaffold to stimulate differentiation of C2C 12 promyoblast towards the osteoblast lineage, the potential of rhBMP-2 scaffolds to induce differentiation and mineralisation of human bone marrow stromal cells was examined in vitro and in vivo.

i) CAM Culture

Culture of human osteoprogenitors on rhBMP-2 encapsulated PLA scaffolds on the chick chorioallantoic membrane model showed that encapsulated rhBMP-2 stimulated human bone marrow stromal cell growth and differentiation in the PLA scaffolds (FIGS. 2B-D). Extensive angiogenesis, as evidenced by new blood vessel growth, was observed on the scaffold/cell constructs from the CAM to the implanted construct over a period of 7 days (FIG. 2A). New cartilage and bone were observed within the chick bone defect as detected by alcian blue and sirius red staining (FIGS. 2B, C) and the use of polarized light microscopy to demonstrate collagen birefringence within the newly formed matrix (FIG. 2D).

Subcutaneous Implantation

Confluent primary human bone marrow cells were trypsinised and seeded (2×10⁵ cells/sample in serum free αMEM) onto PLA scaffolds adsorbed with rhBMP-2 or rhBMP-2 encapsulated PLA scaffolds for 15 hours. Blank (PLA alone) scaffolds were set up in the absence of cells. After 15 hours, constructs were placed in osteogenic media for a further 3 days, prior to subcutaneous implantation into MF1-nu/nu mice (20-24 g, 4-5 weeks old) as previously described⁽³⁶⁾. After 4-6 weeks, the mice were killed and specimens were collected and fixed in 95% ethanol for histochemical analysis.

ii) Subcutaneous Implant Model

Primary human bone marrow cells were seeded onto PLA scaffolds encapsulated with rhBMP-2 and subcutaneous implanted (8 samples) in nude mice for 6 weeks (PLA alone served as a negative control). Poor cell growth and negligible bone matrix synthesis was observed on PLA scaffolds alone (in the absence of rhBMP-2) implanted in nude mice with only fibrous tissue and adipose tissue observed (FIG. 3E). In contrast, rhBMP-2 encapsulated scaffolds promoted human bone marrow stromal cell adhesion, proliferation, differentiation with extensive evidence of new bone matrix deposition as detected by Alcian blue/Sirius red staining for cartilage and bone respectively (FIGS. 3A and 3B). Furthermore, evidence of organised new woven bone within the encapsulated constructs was confirmed by birefringence of collagen using polarized microscopy (FIG. 3B). The efficacy of rhBMP-2 to induce bone formation was confirmed by HBM cell in-growth and bone matrix formation into rhBMP-2 adsorbed PLA scaffolds as detected by Alcian blue and Sirius red staining (FIG. 3C) and (FIG. 3D) Type I collagen staining. Only fibrous tissue and fat tissue were observed in blank (PLA alone) scaffolds (FIG. 3E).

Intra-Peritoneal Implantation

The diffusion chamber (130 μl capacity) model provides an enclosed environment within a host animal to study the osteogenic capacity of skeletally derived cell populations, which resolves the problems of host versus donor bone tissue generation. Cells were recovered by collagenase (Clostridium histolyticum, type IV; 25 U/ml) and trypsin/EDTA digestion. Human bone marrow cells were sealed in diffusion chambers (2×10⁶ cells/chamber) together with PLA porous scaffold encapsulated or adsorbed with or without rhBMP-2. Chambers were implanted intra-peritoneally in MF1-nu/nu mice and after 10 weeks the mice were killed, chambers were removed and examined by X-ray analysis prior to fixation in 95% ethanol at 4° C. Polymer samples were processed undecalcified and sectioned at 5 μm and stained for toluidine blue, type I collagen, osteocalcin and mineralisation by von Kossa.

iii) Diffusion Chamber Model

Recombinant human BMP-2 encapsulated PLA scaffolds seeded with human osteoprogenitor cells, showed morphologic evidence of new bone and cartilage matrix formation as examined by Alcian blue and Sirius red staining (FIGS. 3G, 3J) and by X-ray analysis (FIG. 31) after 10 weeks implantation within diffusion chambers. Metachromatic staining was observed using toluidine blue and collagen deposition and new matrix synthesis was confirmed by birefringence microscopy (FIG. 3H). Cartilage formation could be observed within rhBMP-2 encapsulated PLA scaffolds confirming penetration of human osteoprogenitors through the scaffold constructs (FIG. 3J). No bone formation was observed on cell/PLA scaffold constructs alone (FIG. 3F).

Further aspects and advantages of the invention will be apparent from the foregoing.

1-24. (canceled)

25. A process for the preparation of a polymer composite comprising internally distributed deposition matter wherein the process comprises providing a deposit of deposition matter at the surface of a solid state polymer substrate by fluid phase deposition of discrete particles or dissolved deposition matter by immersion or spraying of solid state polymer substrate with a solution, dispersion or suspension of deposition matter, drying by freezing, evaporation, heating or blotting whereby the deposition matter adsorbs from liquid phase on to the polymer surface and forms an adsorption layer of deposition matter which is intact to solvent and impact effects contacting the surface deposited polymer with a plasticising fluid or a mixture of plasticising fluids under plasticising conditions to plasticise and/or swell the polymer and internally distribute deposition matter, and releasing the plasticising fluid or fluids to obtain polymer composite.

26. A process as claimed in claim 25 wherein deposition matter is present, presented as concentration of deposition matter on polymer, in the range 1×10¹ to 1×10³ ng/mg, or of the order of picograms or nanograms per 5 g polymer, or 1×10⁻¹² to 1×10⁻⁹ wt %.

27. A process as claimed in claim 25 which comprises providing a deposit at the surface of a high surface area polymer substrate.

28. Process as claimed in claim 25 wherein the polymer substrate comprises a powder bed or a high porosity matrix.

29. A process as claimed in claim 25 wherein a deposit comprises a deposition layer of deposition matter on any internal and external exposed surfaces of the polymer substrate, including any exposed surface pores; over the entire surface area or only part or parts thereof.

30. A process as claimed in claim 25 wherein the solid state polymer substrate is obtained by contacting polymer with plasticising fluid or a mixture of plasticising fluids under plasticising conditions to plasticise the polymer, and releasing the fluid in manner to obtain a solid state substrate polymer.

31. A process as claimed in claim 25 carried out in the absence of additional solvent capable of dissolving the deposition matter.

32. A process as claimed in claim 25 wherein immersion is for a time of the order 1 second up to 48 hours.

33. A process as claimed in claim 25 wherein drying is for a time up to 48 hours.

34. A process as claimed in claim 25 wherein plasticising conditions comprise a temperature in the range −200° C. to +500° C.

35. A process as claimed in claim 25 wherein plasticising conditions comprise a pressure from in excess of 1 bar to 10000 bar.

36. A process as claimed in claim 25 wherein the process is carried out for a contact time of surface deposited polymer and plasticising fluid of 20 milliseconds up to 5 minutes.

37. A process as claimed in claim 25 which is carried out without blending.

38. A process as claimed in claim 25 wherein plasticising fluid is selected from carbon dioxide, di-nitrogen oxide, carbon disulphide, aliphatic C₂₋₁₀ hydrocarbons such as ethane, propane, butane, pentane, hexane, ethylene, and halogenated derivatives thereof such as for example carbon tetrafluoride or chloride and carbon monochloride trifluoride, and fluoroform or chloroform, C₆₋₁₀ aromatics such as benzene, toluene and xylene, C₁₋₃ alcohols such as methanol and ethanol, sulphur halides such as sulphur hexafluoride, ammonia, xenon, krypton, and mixtures thereof.

39. A process as claimed in claim 25 wherein deposition material is selected from (pharmaceutical) drugs and veterinary products; agrochemicals as pest and plant growth control agents; human and animal health products; human and animal growth promoting, structural, or cosmetic products including products intended for growth or repair or modelling of the skeleton, organs, dental structure; absorbent biodeposition materials for poisons, toxins.

40. A process as claimed in claim 25 wherein deposition matter alternatively or additionally comprises function enhancing components, including naturally occurring or synthetic or otherwise modified growth promoters, biocompatibilisers, vitamins, proteins, glycoproteins, enzymes, nucleic acid, carbohydrates, minerals, nutrients, steroids, ceramics and the like and functioning matter such as spores, viruses, mammalian, plant and bacterial cells.

41. Process as claimed in claim 25 wherein polymer is selected from: polyesters including poly(lactic acid), poly(glycolic acid), copolymers of lactic and glycolic acid, copolymers of lactic and glycolic acid with poly(ethylene glycol), poly(e-caprolactone), poly(3-hydroxybutyrate), poly(p-dioxanone), poly(propylene fumarate); poly (ortho esters); polyanhydrides; Poly(amino acids); polyacetals; polyketals; polyorthoesters; Polyphosphazenes; azo polymers; synthetic Non-biodegradable Polymers selected from: Vinyl polymers including polyethylene, poly(ethylene-co-vinyl acetate), polypropylene, poly(vinyl chloride), poly(vinyl acetate), poly(vinyl alcohol) and copolymers of vinyl alcohol and vinyl acetate, poly(acrylic acid) poly(methacrylic acid), polyacrylamides, polymethacrylamides, polyacrylates, Poly(ethylene glycol), Poly(dimethyl siloxane), Polyurethanes, Polycarbonates, Polystyrene and derivatives; and Natural Polymers selected from carbohydrates, polypeptides and proteins.

42. A process for the preparation of a polymer composite comprising internally distributed deposition matter wherein the process comprises providing a deposit of deposition matter at the surface of a solid state polymer substrate by fluid phase deposition of discrete particles or dissolved deposition matter by immersion or spraying of solid state polymer substrate with a solution, dispersion or suspension of deposition matter, drying by freezing, evaporation, heating or blotting or by solid phase deposition by powder coating, dusting, rolling or adhering contacting the surface deposited polymer with a plasticising fluid or a mixture of plasticising fluids under plasticising conditions to plasticise and/or swell the polymer and internally distribute deposition matter, and releasing the plasticising fluid or fluids to obtain polymer composite wherein deposition matter is present, presented as concentration of deposition matter on polymer, in the range 1×10¹ to 1×10³ ng/mg, or of the order of picograms or nanograms per 5 g polymer, or 1×10⁻¹² to 1×10⁻⁹ wt %.

43. A process as claimed in claim 42 which comprises providing a deposit at the surface of a high surface area polymer substrate.

44. Process as claimed in claim 42 wherein the polymer substrate comprises a powder bed or a high porosity matrix.

45. A process as claimed in claim 42 wherein a deposit comprises a deposition layer of deposition matter on any internal and external exposed surfaces of the polymer substrate, including any exposed surface pores; over the entire surface area or only part or parts thereof.

46. A process as claimed in claim 42 wherein the solid state polymer substrate is obtained by contacting polymer with plasticising fluid or a mixture of plasticising fluids under plasticising conditions to plasticise the polymer, and releasing the fluid in manner to obtain a solid state substrate polymer.

47. A process as claimed in claim 42 carried out in the absence of additional solvent capable of dissolving the deposition matter.

48. A process as claimed in claim 42 wherein immersion is for a time of the order 1 second up to 48 hours.

49. A process as claimed in claim 42 wherein drying is for a time up to 48 hours.

50. A process as claimed in claim 42 wherein plasticising conditions comprise a temperature in the range −200° C. to +500° C.

51. A process as claimed in claim 42 wherein plasticising conditions comprise a pressure from in excess of 1 bar to 10000 bar.

52. A process as claimed in claim 42 wherein the process is carried out for a contact time of surface deposited polymer and plasticising fluid of 1 millisecond up to 5 hours.

53. A process as claimed in claim 42 which is carried out without blending.

54. A process as claimed in claim 42 wherein plasticising fluid is selected from carbon dioxide, di-nitrogen oxide, carbon disulphide, aliphatic C₂₋₁₀ hydrocarbons such as ethane, propane, butane, pentane, hexane, ethylene, and halogenated derivatives thereof such as for example carbon tetrafluoride or chloride and carbon monochloride trifluoride, and fluoroform or chloroform, C₆₋₁₀ aromatics such as benzene, toluene and xylene, C₁₋₃ alcohols such as methanol and ethanol, sulphur halides such as sulphur hexafluoride, ammonia, xenon, krypton, and mixtures thereof.

55. A process as claimed in claim 42 wherein deposition material is selected from (pharmaceutical) drugs and veterinary products; agrochemicals as pest and plant growth control agents; human and animal health products; human and animal growth promoting, structural, or cosmetic products including products intended for growth or repair or modelling of the skeleton, organs, dental structure; absorbent biodeposition materials for poisons, toxins.

56. A process as claimed in claim 42 wherein deposition matter alternatively or additionally comprises function enhancing components, including naturally occurring or synthetic or otherwise modified growth promoters, biocompatibilisers, vitamins, proteins, glycoproteins, enzymes, nucleic acid, carbohydrates, minerals, nutrients, steroids, ceramics and the like and functioning matter such as spores, viruses, mammalian, plant and bacterial cells.

57. Process as claimed in claim 42 wherein polymer is selected from: polyesters including poly(lactic acid), poly(glycolic acid), copolymers of lactic and glycolic acid, copolymers of lactic and glycolic acid with poly(ethylene glycol), poly(e-caprolactone), poly(3-hydroxybutyrate), poly(p-dioxanone), poly(propylene fumarate); poly(ortho esters); polyanhydrides; Poly(amino acids); polyacetals; polyketals; polyorthoesters; Polyphosphazenes; azo polymers; synthetic Non-biodegradable Polymers selected from: Vinyl polymers including polyethylene, poly(ethylene-co-vinyl acetate), polypropylene, poly(vinyl chloride), poly(vinyl acetate), poly(vinyl alcohol) and copolymers of vinyl alcohol and vinyl acetate, poly(acrylic acid) poly(methacrylic acid), polyacrylamides, polymethacrylamides, polyacrylates, Poly(ethylene glycol), Poly(dimethyl siloxane), Polyurethanes, Polycarbonates, Polystyrene and derivatives; and Natural Polymers selected from carbohydrates, polypeptides and proteins.

58. A polymer composite when obtained by the process of claim 25.

59. The use of a polymer composite or a scaffold thereof prepared by the process of claim 25, for drug delivery, in bioremediation, as a biocatalyst or biobarrier for human or animal or plant matter, as a structural component comprising the polymer and optional additional synthetic or natural metal, plastic, carbon or glass fibre mesh, scrim, rod or like reinforcing for medical or surgical insertion, for insertion as a solid monolith into bone or tissue, as fillers or cements for wet insertion into bone or teeth or as solid aggregates or monoliths for orthopaedic implants such as pins, or dental implants such as crowns.
